SEQUENCE LISTING (14 sequences)

SEQ ID NO: 1 - DNA, 22 bp, Artificial Sequence
  Description of Artificial Sequence Synthetic oligonucleotide
tgtcctccaatttgtcctggtt

SEQ ID NO: 2 - DNA, 23 bp, Artificial Sequence
  Description of Artificial Sequence Synthetic oligonucleotide
gcagcaggatgaagaggaatatg

SEQ ID NO: 3 - DNA, 23 bp, Artificial Sequence
  Description of Artificial Sequence Synthetic oligonucleotide
cgctggatgtgagtcggaacctt

SEQ ID NO: 4 - DNA, 21 bp, Artificial Sequence
  Description of Artificial Sequence Synthetic oligonucleotide
aaggttccgacttctgcggcg

SEQ ID NO: 5 - DNA, 20 bp, Artificial Sequence
  Description of Artificial Sequence Synthetic oligonucleotide
cgctggatgtgtctgcggcg

SEQ ID NO: 6 - DNA, 28 bp, Artificial Sequence
  Description of Artificial Sequence Synthetic oligonucleotide
gaaattctcagaatttctgaaaggttac

SEQ ID NO: 7 - DNA, 28 bp, Artificial Sequence
  Description of Artificial Sequence Synthetic oligonucleotide
cctctgggctaataggactacttctaat

SEQ ID NO: 8 - DNA, 24 bp, Artificial Sequence
  Description of Artificial Sequence Synthetic oligonucleotide
yycttgcctgtccagagtcggaac

SEQ ID NO: 9 - DNA, 22 bp, Artificial Sequence
  Description of Artificial Sequence Synthetic oligonucleotide
ctcgcctgtccagagtcggaac

SEQ ID NO: 10 - DNA, 23 bp, Artificial Sequence
  Description of Artificial Sequence Synthetic oligonucleotide
gttccgactggatctgctcttac

SEQ ID NO: 11 - DNA, 20 bp, Artificial Sequence
  Description of Artificial Sequence Synthetic oligonucleotide
cgctggatgtgagtcggaac

SEQ ID NO: 12 - DNA, 18 bp, Artificial Sequence
  Description of Artificial Sequence Synthetic oligonucleotide
gttccgacttctgcggcg

SEQ ID NO: 13 - DNA, 12 bp, Artificial Sequence
  Description of Artificial Sequence Synthetic oligonucleotide
agtcggaacctt

SEQ ID NO: 14 - DNA, 12 bp, Artificial Sequence
  Description of Artificial Sequence Synthetic oligonucleotide
aaggttccgact

(19) United States
(12) Patent Application Publication
(10) Pub. No.: US 2007/0026423 A1
(43) Pub. Date: Feb. 1, 2007

(54) Method and test kit for the detection of target nucleic acids

(76) Inventors: Thomas Koehler, Borsdorf (DE); Anne-Katrin Rost, Borsdorf (DE)

Correspondence Address: JORDAN AND HAMBURG LLP, 122 EAST 42ND STREET, SUITE 4000, NEW YORK, NY, 10168, US

(21) Appl. No.: 11/393,912

(22) Filed: Mar. 16, 2006

Related U.S. Application Data
(60) Provisional application No. 60/668,454, filed on Apr. 4, 2005.

(30) Foreign Application Priority Data
Mar. 16, 2005 (DE) 10 2005 000 021.5

(51) Int. Cl. C12Q 1/68

(52) U.S. Cl. 435/6

(57) ABSTRACT

This invention concerns a method for the detection of target nucleic acids or analoga to nucleic acids (targets), wherein at least one pair of labeled oligonucleotides or analoga to oligonucleotides (probes) are used, characterised in that the upstream probe proportionally hybridises to the target with its 5′ end and proportionally hybridises to the 5′ end of the downstream probe with its 3′ end, as well as the 3′ end of the downstream probe proportionally hybridises to the target again, this resulting in a triplex structure built up between the target nucleic acid and the probe pair. This invention moreover relates to a test kit required to carry out the method.

This invention relates to a method for the detection of target nucleic acids or analoga to nucleic acids (targets), in which at least one pair of labeled oligonucleotides or analoga to oligonucleotides (probes) are utilised, wherein the upstream probe proportionally hybridises to the target with its 5′ end and proportionally hybridises to the 5′ end of the downstream probe with its 3′ end, as well as the 3′ end of the downstream probe proportionally hybridises to the target again, this resulting in the formation of a triplex structure between the target nucleic acid and the probe pair. This invention moreover relates to a test kit required to carry out the method.

This invention relates to probe pairs used to homogeneously detect heteropolymer target sequences for the qualitative and quantitative “real-time” or endpoint detection of heteropolymer nucleic acids or analoga to nucleic acids after they have enzymatically been amplified. Possible fields of application include molecular biology, qualitative and quantitative analysis of genes, gene expressions, viral and microbial pathogenes, respectively, genetically modified organisms found in food, in particular for the application in individualised medical diagnostics, functional genome analysis, clinical pharmacology, pharmacogenetics, forensics, food and environmental analyses, as well as for the ultra-sensitive detection of proteins by means of immuno-PCR.

Enzymatic amplification procedures are often used for molecular diagnostics of qualitative gene changes and polymorphisms, as well as quantitative diagnostics of pathogenes, where in particular the analyte concentration is extremely low. Examples for this include such methods as Polymerase Chain Reaction (PCR, U.S. Pat. Nos. 4,683,195 A, 4,683,202 A, EP 0 200 362 A1, EP 0 201 184 A1; Hoffmann-La Roche), Ligase Chain Reaction (LCR, Abbott Diagnostics, North Chicago, Ill., USA), Strand Displacement Amplification (SDA, Walker et. al. [1993], PCR Methods Appl. 3: 1-6, Becton-Dickinson Research Center) and Transcription-Mediated Amplification (TMA, Gen-Probe Inc., San Diego, Calif.). All the methods mentioned constitute in vitro methods for exponential amplification of the target nucleic acid to be measured.

Homogeneous assays allow to measure in vitro synthesis of PCR products in “real-time”, i.e. to measure it directly in the PCR reaction recipients used. Procedures suited for this according to the state of the art include, but are not limited to “Fluorescence resonance energy transfer (FRET)” based “Dye-labeled oligonucleotide ligation (DOL)” methods [Chen, X., Livak, K. J. & Kwok, P. Y. Genome Res. 8, 549-556 (1998), Landegren, U., Nilsson, M. & Kwok, P. Y. Genome Res. 8, 769-776 (1998). U.S. Pat. No. 6,027,889; U.S. Pat. No. 6,268,148] the 5′-Exonuclease or TaqMan-Assay [Holland, P. M., Abramson, R. D., Watson, R. & Gelfand, D. H. Proc. Natl. Acad. Sci. U.S.A. 88, 7276-7280 (1991), Didenko, V. V. Biotechniques 31, 1106-1 (2001); U.S. Pat. No. 5,210,015; U.S. Pat. No. 5,487,972; EP 0 919 565 A2; WO 92/02638] as well as the “Adjacent hybridization probes” or Dual probes methods (U.S. Pat. No. 5,532,129; U.S. Pat. No. 5,565,322, U.S. Pat. No. 5,914,230, EP 1 389 638 A1). The so-called non FRET-based detections methods comprise such methods as “Molecular beacons” [Kwiatkowski, R. W., Lyamichev, V., de Arruda, M. & Neri, B. Mol. Diagn. 4, 353-364 (1999), U.S. Pat. No. 5,989,823], the “Self priming probes” (U.S. Pat. No. 5,866,336), the LightUp method (EP 1 357 185 A1, U.S. Pat. No. 6,461,871 B1) as well as the PCR-independent Invader-Assay [Cooksey, R. C., Holloway, B. P., Oldenburg, M. C., Listenbee, S. & Miller, C. W. Antimicrob. Agents Chemother. 44, 1296-1301 (2000)].

To carry out nucleic acid quantification reactions, the homogeneous and very robust 5′-3′ exonuclease assay is often used. This method is based on a conventional PCR principle known to the man skilled in the art, where a single-strand DNA probe complementary to the amplicon single strand and contained in the reagent mix hybridises to the target between the primer binding sites. This probe, which is labeled with a reporter dye at the 5′ end and with a quencher dye at the 3′ end, is hydrolysed by the 5′-3′ exonuclease activity of the Taq polymerase as a result of primer extension, this resulting in the neutralisation of the quench effect and in the creation of a fluorescence signal proportional to the amount of the target product.

The above-mentioned methods are principally based on the formation of duplex structures between the target sequence and the probe(s) labeled and have the disadvantage that there is always only one fluorescence measuring pulse that can maximally be created per enzymatically synthesised amplification product. Another disadvantage is that the probe formats used are either suited for the dual probes assay or the 5′-3′ exonuclease assay, but never for both detection formats at the same time. The detection method based on adjacent hybridisation probes” has moreover the disadvantage that the probes utilised for such methods require very long (>40 bp) hybridisation areas, this making its application particularly complicated, when hyper-variable targets containing short preservation areas only, in particular RNA targets, are to be detected. Further disadvantages are that particularly the TaqMan method, which is used very often, is not very robust and thus very sensitive to perturbations emanating from inhibiting substances contained in the sample. Moreover, most of the detection methods named above show the disadvantage that the probes used must feature a 3′OH blockage modification in order to avoid an undesirable elongation of the probe during the PCR process.

That's why this invention has the function of eliminating the disadvantage inherent to the solutions described representing the current state of technology.

The problem has been solved by a method of detecting target nucleic acids or analoga to nucleic acid (targets), in which at least one pair of labeled oligonucleotides or analoga to oligonucleotides (probes) are utilised, wherein the upstream probe proportionally hybridises to the target with its 5′ end and proportionally hybridises to the 5′ end of the downstream probe with its 3′ end, as well as the 3′ end of the downstream probe proportionally hybridises to the target again, this resulting in a triplex structure formed between the target nucleic acid and the probe pair. An optimum hybridisation of the probes to the target can only be reached using a pair of probes, however not by means of individual probes. The sequence sections of the upstream and downstream probes enabled in proportion to the target hybridisation will preferably hybridise to adjacent sequences of the target, and at the same time, a stabilising “stem structure” is formed between the 3′ end of the upstream probe and the 5′ end of the downstream probe.

The method according to the invention has the advantage that labels can be located both at the 5′ end and the 3′ end of the two probes so that there is a total of 4 different loci available for different label combinations.

The triplex structure formed between the probe pair and the target is either directly enabled to form a measuring signal as a result of the hybridisation event, or a measuring signal is generated, for example by an enzyme also contained in the homogeneous assay, preferably a polymerase, as late as after partial hydrolysis of the probe pair has taken place.

The present invention moreover relates to a pair of probes for the detection of target nucleic acids (targets), wherein the 5′ end of the upstream probe is sufficiently complementary to a sub-sequence of the target, and that the 3 end of this probe is sufficiently complementary to the 5′ end of the downstream probe, as well as that the 3′ end of the downstream probe is again sufficiently complementary to the 5′ end of the upstream probe and that the 3′ end of this probe is again sufficiently complementary to another sub-sequence of the target.

Surprisingly, it has turned out that either one of the probes or both probes—in contrast with the current state of technology—are not required to feature a 3′ OH blockage.

All oligonucleotides or all analoga to oligonucleotides that are able to develop a stem structure can be used as a probe pair. For example, the stem structure can be developed through the sub-sequences 5′-AGTCGGAACCTT-3′ and 5′-AAGGTTCCGACT-3′.

According to this invention, one of the probes or both probes bear a label at the 5′ end and/or at the 3′ end. Fluorescence dyes and/or quencher dyes can be used as a label. Moreover, it is possible to mark one probe with each one donor dye and one acceptor dye at its 3′ end and the other probe at its 5′ end.

Hence, a universally applicable and robust probe format has been developed which is suitable for several FRET-based detection methods and which compensates for the disadvantage of the formation of duplex structures for the measurement of heteropolymer target sequences by the fact that up to 2 measuring pulses can be generated per probe-target-complex, and that no 3′ OH blockage is required, as well as which compensates for the disadvantage of the “Adjacent hybridisation probes” method to the effect that from now on, shorter, preserved genome section can be made usable for probe hybridisation, improving in particular the detection of variable target sequences.

According to this invention, the method of manufacturing the probe pair comprises the following operations:
Breakdown of a known or unknown oligonucleotide probe sequence compatible with the target with a preferable length of 15-40 bases into two partial probe sequences;
Replenishment of the partial probe sequences by one sub-sequence each developing a stem structure between the probes of the probe pair according to claim no. 8;
chemical coupling of a molecule or atom, able to generate a measurable, preferably quantifiable signal (labels).

Preferably, both the partial probe sequences hybridised to the target in relation to each other and the probes of the probe pair themselves show similar Tₘ, values each time.

The method for the detection of target nucleic acids or analoga to nucleic acids (targets) and the probes according to this invention are utilised in a test kit, which is also the object of this invention. This test kit comprises at least one pair of probes as described in this invention, at least one heteropolymer target sequence as well as a set of reagents known to a person skilled in the art. In addition, the test kit may also comprise at least one enzyme.

The probe pair and/or the method for the detection of target nucleic acids and/or the test kit as described by this invention are suited for qualitative and/or quantitative detection of any heteropolymer target nucleic acids or analoga to nucleic acids.

Principle of the Invention

The principle of this invention is described in FIG. 1. A pair of probes according to this invention is composed of two probes, wherein each individual probe is always composed of 2 different functional sequence sections, a probe section complementary to the target sequence and a probe section developing a stem structure to the related second probe. A sufficient hybridisation of probes is only possible for the probe pair, however not for the individual probe under the chosen reaction conditions. There are at least 4 loci per probe pair designated by M1-M4 for labeling, which preferably uses fluorescence dyes and/or quencher dyes. In a preferred embodiment of the invention, probe 1 or probe 2, or probe 1 and probe 2 are labeled with one or two reporter dyes each, for examples with the fluoresceine dyes or rhodamine dyes FAM, VIC, JOE, HEX, NED, Yakima Yellow, ROX Cy3, Cy5 or DYXL, and one or two quencher dyes, such as TAMRA, DABCYL, DABSYL, ElleQuencher or DarkQuencher. In another preferred embodiment of this invention, the two probes are preferably labeled in the stem area (M2+M3) with a donor dye and an acceptor dye each, such as fluoresceine, LCRed640 or LCRed705. Table 1 shows some of the combinations possible for labeling.

The probe pair is developed by breaking down a known oligonucleotide probe sequence compatible with the selected amplification system, such as a TaqMan probe, into two partial probe sequences preferably having a similar Tₘ value and by replenishing the partial probe sequences with suitable, complementary partial sequences developing a later stem structure. The probe pairs are manufactured by means of a method of conventional oligonucleotide solid phase synthesis followed by chemical coupling of the chromophoric dyes preferably to be used, which is known to a person skilled in the art.

TABLE 1
Examples of different possible combinations of labels for the probe pair
according to this invention (FIG. 1). Designations: reporter (R),
quencher (Q), donor (D), acceptor (A), no label (Ø)

| Variety | M1 | M2 | M3 | M4 |
|---|---|---|---|---|
| 1 | R | Q | Ø | Ø |
| 2 | Ø | Ø | R | Q |
| 3 | R | Q | R | Q |
| 4 | Ø | D | A | Ø |
| 5 | R | Ø | Q | Ø |
| 6 | R | Q | Ø | Q |
| 7 | Ø | Q | R | Q |

Surprisingly, it has turned out that, in contrast with other probe formats of the current state of technology, a 3′ OH blockage is required neither for probe 1 nor probe 2 of the probe pair according to this invention when using the pair of probes.

A possible embodiment of this invention is constituted by the utilisation of the probe pair according to this invention in enzymatic amplification reactions known to a person skilled in the art, such as the polymerase chain reaction (PCR).

The nature of this invention thus lies with a combination of known elements and new solution methods, influencing each other, resulting in a utility advantage and yielding the success intended by the action of their new overall effect, this being characterised in that a triplex structure is available now with the probe pairs developing the target sequence for a sensitive homogeneous detection of selected nucleic acids contained in biological substances.

The use of the probe pairs according to this invention is constituted by their utilisation for a validated, standardised detection of the target sequence DNA or RNA in preferably biological experimental substances, wherein individual oligonucleotides, in particular probes and primer, can also be usable, when taken out of the system, separately for PCR-independent enzymatic amplification reactions, even in connection with other detection techniques, if so required.

### FIGURES

FIG. 1: Functional principle of the probe pair according to this invention

FIGS. 2 to 7: Utilisation of a probe pair according to this invention for the measurement of hepatitis B viruses (HBV) through sequence sections of the surface antigene, HBsAg Gene by means of quantitative PCR (2S). Comparison of the validity with a similar quantitative TaqMan protocol (2T) by using identical target sequences.

2S1: Real-time PCR amplification of a HBV standard DNA by utilising the probe pair according to this invention, saturation curves

2S2: Real-time PCR amplification of a HBV standard DNA by utilising the probe pair according to this invention, standard reference curve, slope=−3.498; Intercept=45.028, R2=0.997

2S3: Real-time PCR amplifications of a cleaned clinical HBV sample, from which the DNA had been washed out before, by utilising the probe pair according to this invention, saturation curves

2T1: Real-time PCR amplification of an HBV standard DNA by utilising a comparable TaqMan protocol, saturation curves

2T2: Real-time PCR amplification of an HBV standard DNA by utilising a comparable TaqMan protocol, standard reference curve, slope=−3.209; Intercept=40.046, R2=0.999

2T3: Real-time PCR amplification of a clinical HBV sample, from which the DNA had been washed out before, by utilising a comparable TaqMan protocol, saturation curves

### EMBODIMENTS

### Embodiment 1: Probe Pair According to the Invention for Measuring Hepatitis B Viruses (HBV) via Sequence Sections of the Surface Antigene, HBsAg Genes using Quantitative PCR

Oligonucleotides used (GeneBank Accession M20919):

| | |
|---|---|
| SEQ ID No. 1 Primer1_HBV [233] | 5′ 520-541 3′ |
| | 5′-TGTCCTCCAATTTGTCCTGGTT-3′ |
| SEQ ID No. 2 Primer2_HBV [234] | 5′ 570-592 3′ |
| | 5′-GCAGCAGGATGAAGAGGAATATG-3′ |
| SEQ ID No. 3 Probe 1 [393] | 5′ 544-554 3′ |
| | 5′-FAM-CGCTGGATGTGAGTCGGAACCTT-3′-BHQ1 |
| | Tₘ = 79.1° C. |
| SEQ ID No. 4 Probe 2 [394] | 5′ 555-563 3′ |
| | 5′-AAGGTTCCGACTTCTGCGGCG-3′ |
| | Tₘ = 79.2° C. |

The Tₘ values were calculated using Oligo Primer Analysis Software vers. 6.23 (Molecular Biology Insights, Inc.) according to the % GC rule. The sequences underlined represent the related complementary, stem-forming sections of the probe pair according to the invention.

Amplification:

The HBsAg Gene region amplified by means of SEQ ID no. 1 and SEQ ID no. 2 resulted in an amplicon with a length of 73 bp. The following optimised protocol was chosen to utilise the probe pair according to the invention for a 25-μl PCR mix (table 2):

TABLE 2
Reagents used and concentrations optimised for the utilisation
of the probe pair according to the invention

| | 25 μl mix | | | |
|---|---|---|---|---|
| | Volume | Volume | | |
| Reactive components in the | per mix | Mastermix | Mix | |
| mix | (μl) | (μl) | concentration | |
| H₂O (PCR grade) | 10.7 | 128.4 | — | |
| 10x ROX buffer | 2.5 | 30.0 | 1x | |
| MgCl₂ (50 mM) | 3.5 | 42.0 | 7 | mM |
| Nucleotide mix (2.5 mM | 2.0 | 24.0 | 0.2/ | |
| dATP, 2.5 mM dCTP, 2.5 | | | 0.4 | mM (dUTP) |
| mM dGTP, 5 mM dUTP) | | | | |
| SEQ ID No. 1 (15 μM) | 0.5 | 6.0 | 300 | nM |
| SEQ ID No. 2 (15 μM) | 0.5 | 6.0 | 300 | nM |
| SEQ ID No. 3 (2.0 μM) | 2.5 | 30.0 | 200 | nM |
| SEQ ID No. 4 (2.0 μM) | 2.5 | 30.0 | 200 | nM |

| | | | |
|---|---|---|---|
| HBV Standard DNA (HBV- | — | — | Refer to table 3 |
| DNA coated 8 tubes/strip) | | | |
| AmpliTaq Gold DNA | 0.3 | 3.6 | 1.5 |
| Polymerase (5 U/μl, Applied | | | |
| Biosystems) | | | |

TABLE 3
Concentrations of the HBV standards used, calibrated against WHO
reference material and related to 1 ml sample material to be examined
(serum or plasma).

| Tube | | |
|---|---|---|
| no. | HBV Standard DNA [IU/ml] | HBV Standard DNA [copies/ml] |
| 1 | 20,000,000 | 100,000,000 |
| 2 | 2,000,000 | 10,000,000 |
| 3 | 200,000 | 1,000,000 |
| 4 | 50,000 | 250,000 |
| 5 | 20,000 | 100,000 |
| 6 | 5,000 | 25,000 |
| 7 | 1,000 | 5,000 |
| 8 | 200 | 1,000 |

The reagents from table 2 have initially been put together to form a mastermix sufficient for 12 PCR mixes. 25-μl aliquote of the mastermix were transferred by pipette into the 8 reaction recipients coated with different concentrations of standard HBV-DNA (Roboscreen, DE 198 40 531) as well as into the 3 reaction recipients not coated with standard HBV-DNA (PCR blank values). The concentrations of the standards used are represented in table 3. Amplification and detection of the PCR products were carried out in real-time using ABI PRISM 7000 Sequence Detection Systems (Applied Biosystems). The following thermoprofile was chosen:

| | | |
|---|---|---|
| Initial denaturalisation | | 95° C. 10:00 min |
| 40 cycles (3 step PCR) | Denaturalisation | 95° C. 00:30 min |
| | Annealing | 45° C. 00:15 min |
| | Extension | 57° C. 01:15 min |

Result:

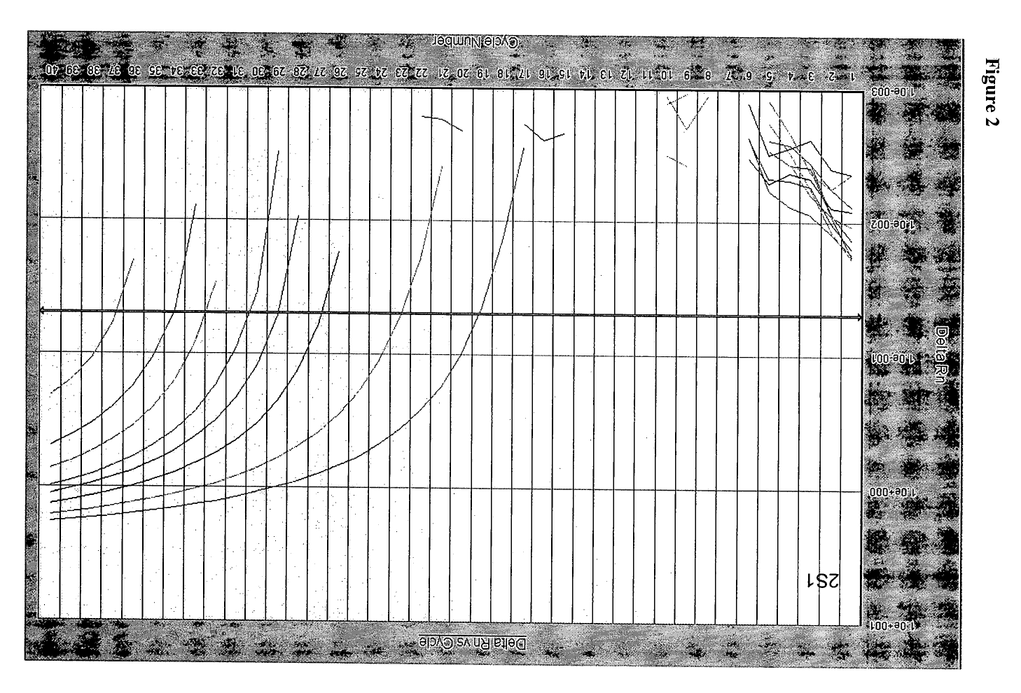

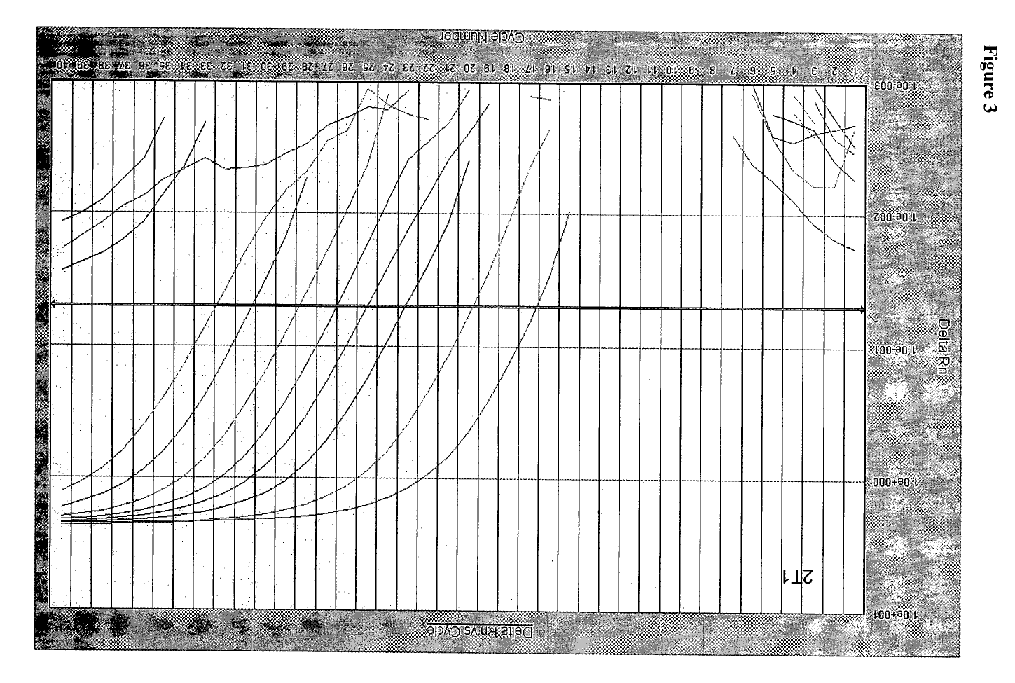

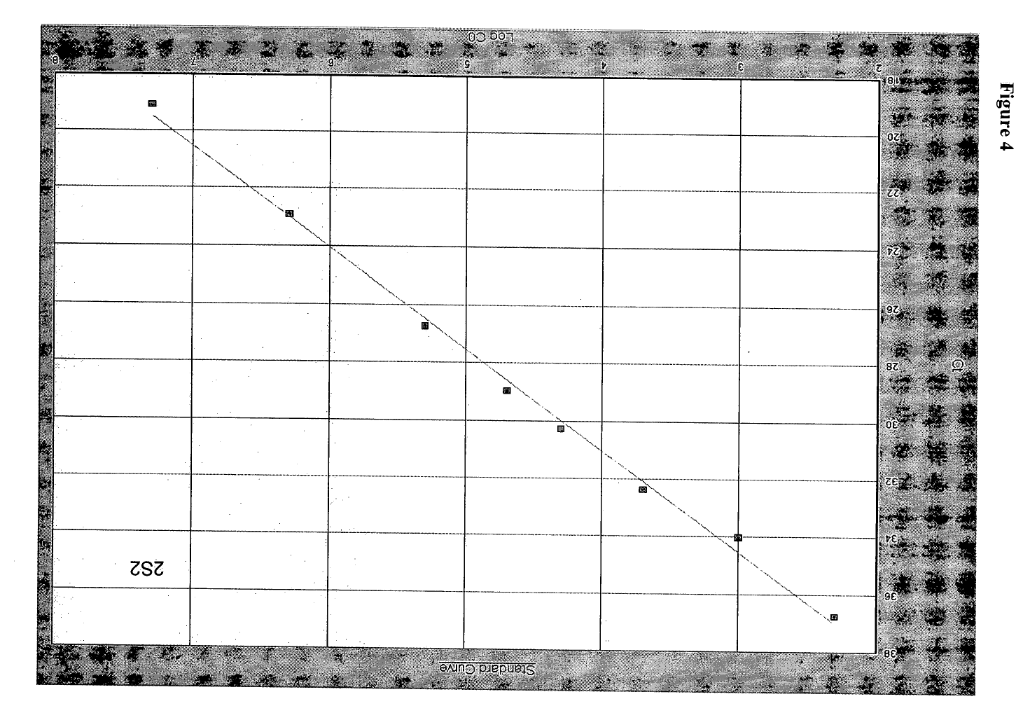

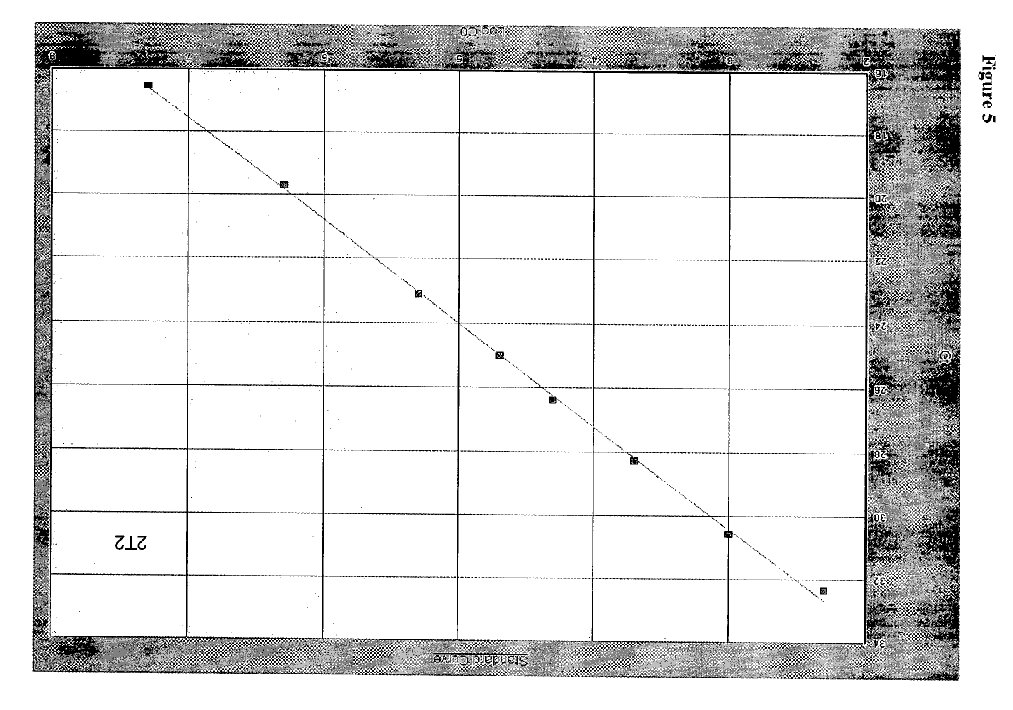

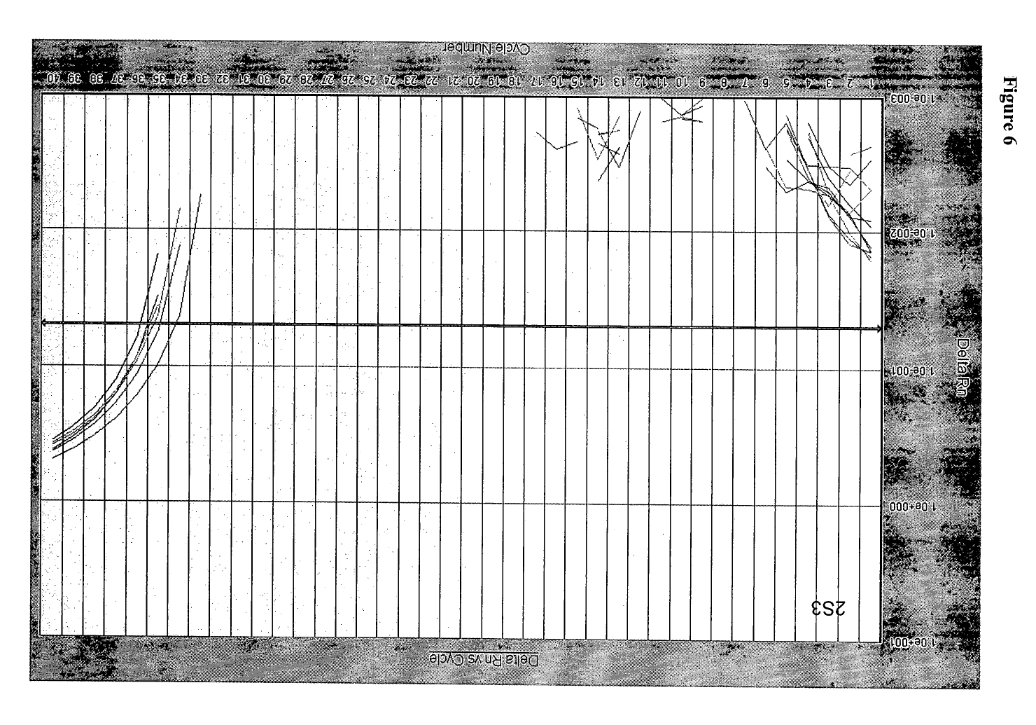

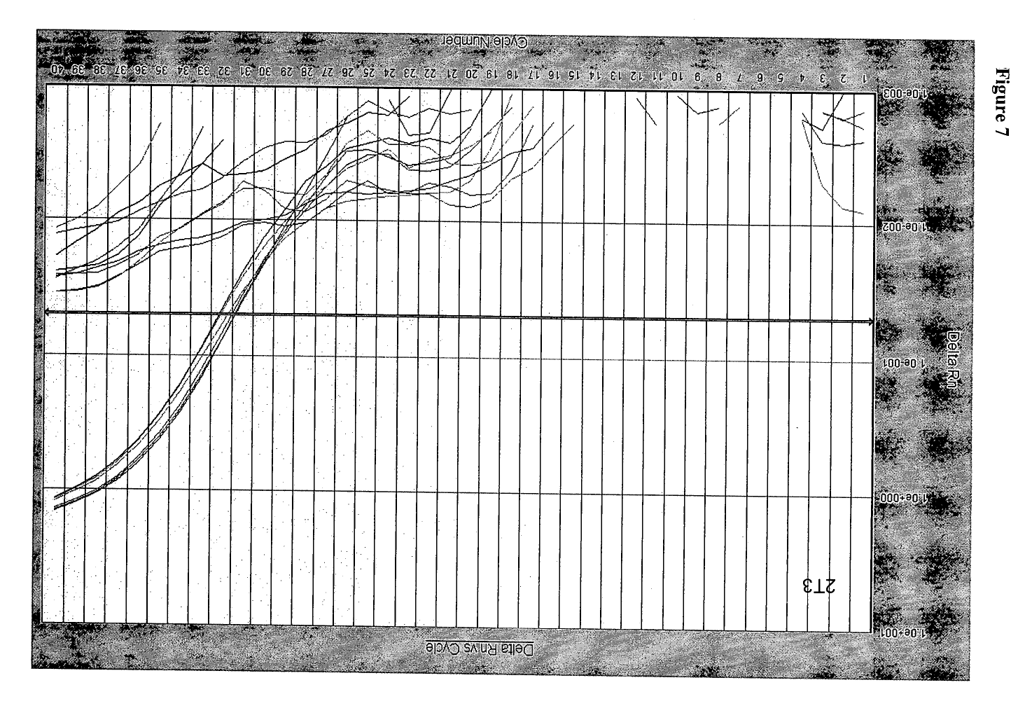

Whilst the PCR blank value did not yield any exponential amplification signal, i.e. there was no contamination of the detection system, a desired amplification signal in the form of a saturation curve was detected (FIG. 2S1) in all standard HBV DNA coated reaction recipients. Evaluation of the curves made it possible to set up a standard reference curve (FIG. 2S2), which comes up to the expectations of a person skilled in the art in terms of the evaluation parameters slope, intercept and linear regression (R2). The quality of these data is comparable with those of the well-established TaqMan method applied in embodiment 2 (embodiment 2, FIGS. 2T1 and 2T2).

### Embodiment 2: TagMan Method for Measuring Hepatitis B Viruses (HBV) via Sequence Sections of the Surface Antigene, HBsAg Genes by Means of Quantitative PCR

Oligonucleotides utilised in addition to the SEQ ID no. 1 and SEQ ID no. 2 (embodiment 1) (GeneBank Accession M20919):

| | |
|---|---|
| SEQ ID No. 5 HBV_Prob [235] | 5′ 544-563 3′ |
| | 5′-FAM-CGCTGGATGTGTCTGCGGCG-3′-TAMRA |
| | Tₘ = 81.4° C. |

The Tₘ values were calculated using Oligo Primer Analysis Software vers. 6.23 (Molecular Biology Insights, Inc.) according to the % GC rule.

Amplification:

The HBsAg Gene region amplified by means of SEQ ID no. 1 and SEQ ID no. 2 resulted in an amplicon with a length of 73 bp. For real-time detection of the amplicon using SEQ ID no. 5, the following optimised protocol for a 25-μl PCR mix was chosen (table 4): The following protocol optimised for a 25-μl PCR mix (table 4) was chosen for real-time detection of the amplicon using SEQ ID no. 5:

TABLE 4
Reagents used and concentrations optimised for the TaqMan protocol

| | 25 μl mix | | | |
|---|---|---|---|---|
| | | Volume | | |
| | Volume | per | | |
| Reactive components in the | per mix | Mastermix | | |
| mix | (μl) | (μl) | Mix concentration | |
| H₂O (PCR grade) | 16.2 | 194.4 | — | |
| 10x ROX buffer | 2.5 | 30.0 | 1x | |
| MgCl₂ (50 mM) | 1.5 | 18.0 | 3 | mM |
| Nucleotide mix (2.5 mM | 2 | 24.0 | 0.2/ | |
| dATP, 2.5 mM dCTP, 2.5 | | | 0.4 | mM (dUTP) |
| mM dGTP, 5 mM dUTP) | | | | |
| SEQ ID no. 1 (15 μM) | 0.5 | 6.0 | 300 | nM |
| SEQ ID no. 2 (15 μM) | 0.5 | 6.0 | 300 | nM |
| SEQ ID no. 5 (2.0 μM) | 1.5 | 18.0 | 120 | nM |

| | | | |
|---|---|---|---|
| HBV Standard DNA (HBV- | — | — | Refer to table 3 |
| DNA coated 8 tubes/strip) | | | |
| AmpliTaq Gold DNA | 0.3 | 3.6 | 1.5 |
| Polymerase (5 U/μl, Applied | | | |
| Biosystems) | | | |

The reagents from table 4 have initially been put together to form a mastermix sufficient for 12 PCR mixes. 25-μl aliquote of the mastermix were transferred by pipette into the 8 reaction recipients coated with different concentrations of standard HBV-DNA (Roboscreen, DE 198 40 531) as well as into the 3 reaction recipients not coated with standard HBV-DNA (PCR blank values). The concentrations of the standards used are represented in table 3. Amplification and detection of the PCR products were carried out in real-time using ABI PRISM 7000 Sequence Detection Systems (Applied Biosystems). The following thermoprofile was chosen:

| | | |
|---|---|---|
| Initial Denaturalisation | | 95° C. 10:00 min |
| 40 cycles (2 step PCR) | Denaturalisation | 95° C. 00:15 min |
| | Annealing/Extension | 59° C. 01:15 min |

Result:

Whilst the PCR blank value did not yield any exponential amplification signal, i.e. there was no contamination of the detection system, a desired amplification signal in the form of a saturation curve was detected (FIG. 2T1) in all standard HBV DNA coated reaction recipients. Evaluation of the curves made it possible to set up a standard reference curve (FIG. 2T2), which comes up to the expectations of a person skilled in the art in terms of the evaluation parameters slope, intercept and linear regression (R2).

### Embodiment 3 Practical Comparison of the Method According to this Invention with the Established TaqMan Method using Clinical Samples Containing HBV Viruses

Clinical Sample:

The clinical HBV-positive sample used was HBV reference plasma from Paul-Ehrlich-Institut (50,000 IU/ml (Lot #1872/01, genotype D, subtype ayw2/3). The sample was diluted with HBV negative plasma from a blood donor not affected by HBV down to 1000 IU/ml. The HBV negative plasma was used as negative cross-check substance.

Extraction of Nucleic Acid from the Clinical Sample:

To extract the DNA from the HBV-positive sample and the negative cross-check substance, an RTP® DNA/RNA Virus Mini Kit (Roboscreen GmbH) was used according to the manufacturer instructions. The extraction was carried out in triple mixes from 200 μl of the HBV-positive plasma and from the HBV-negative plasma, respectively. Elution of the extracted DNA was effected by means of 60 μl of the elution buffer contained in the kit per sample plasma.

Amplification:

The HBsAg Gene region amplified by means of SEQ ID no. 1 and SEQ ID no. 2 resulted in an amplicon with a length of 73 bp. For “real-time” quantification of the HBV-copies using the method according to this invention and the TaqMan control method, the protocols explained in embodiments 1 and 2 were used. The mastermixes have been represented as shown in table 5.

TABLE 5
Reagents used and manufacture of master-mixes

| Reactive | Probe pair | | TaqMan | |
|---|---|---|---|---|
| components in | Vol. per mix | Mastermix | Vol. per mix | Mastermix |
| the mix | (μl) | (μl) | (μl) | (μl) |
| H₂O (PCR grade) | 7.7 | 100.1 | 11.2 | 145.6 |
| 10x ROX-buffer | 2.5 | 32.5 | 2.5 | 32.5 |
| MgCl₂ (50 mM) | 1.5 | 19.5 | 1.5 | 19.5 |
| Nucleotide mix (2.5 | 2.0 | 26.0 | 2.0 | 26.0 |
| mM dATP, 2.5 mM | | | | |
| dCTP, 2.5 mM | | | | |
| dGTP, 5 mM dUTP) | | | | |
| SEQ ID No. 1 | 0.5 | 6.5 | 0.5 | 6.5 |
| (15 μM) | | | | |
| SEQ ID No. 2 | 0.5 | 6.5 | 0.5 | 6.5 |
| (15 μM) | | | | |
| SEQ ID No. 3 | 2.5 | 32.5 | — | — |
| (2.0 μM) | | | | |
| SEQ ID No. 4 | 2.5 | 32.5 | — | — |
| (2.0 μM) | | | | |
| SEQ ID No. 5 | — | — | 1.5 | 19.5 |
| (2.0 μM) | | | | |
| Extracted HBV | 5 | — | 5 | — |
| sample DNA | | | | |
| AmpliTaq Gold | 0.3 | 3.9 | 0.3 | 3.9 |
| DNA Polymerase | | | | |
| (5 U/μ) | | | | |

The reagents of table 5 were initially put together to form a mastermix sufficient for 13 PCR mixes, and transferred to the related number (12 each) of 0.2 ml DNA sample reaction recipients (Roboscreen) in 20-μl aliquotes. Both the HBV positive and HBV negative, clinical samples were amplified using the two methods compared in double identification, i.e. 6 identical PCR mixes per run were prepared using an identical sample (1000 IU/ml). Amplification and detection of the PCR products were carried out in real-time using an ABI PRISM 7000 Sequence Detection Systems (Applied Biosystems). The thermoprofile were selected as in embodiment 1 (probe pair) and embodiment 2 (TaqMan).

Results:

Whilst the negative controls and the PCR blank value (5 μl PCR grade H₂O instead of extracted HBV-DNA sample from PCR mix) resulted in no exponential amplification signal, i.e. there was no contamination of the detection system at all, all HBV-positive samples could clearly be amplified both by means of the method according to the invention (FIG. 2S3) and by using the TaqMan comparison method (FIG. 2T3). Quantitative analysis has turned out (table 6) that the detection of HBV copies using the probe pair according to this invention was characterised by a significant increase in efficiency by 33% in contrast with the TaqMan method (on average: 744 out of 1000 IU/ml compared with 417 out of 1000 IU/ml). The reason for this could be a higher robustness of the method in contrast with the TaqMan method, which is known to be prone to interference.

TABLE 6
Quantitative measurement results obtained by the two methods

| | IU/ml | |
|---|---|---|
| Measurement no. | TaqMan | IU/ml probe pair |
| 1 | 523 | 1316 |
| 2 | 527 | 673 |
| 3 | 300 | 455 |
| 4 | 466 | 585 |
| 5 | 394 | 822 |
| 6 | 291 | 613 |
| Mean value | 417 | 744 |
| Standard deviation (SD) | 106 | 305 |

### DEFINITIONS

Labeled Oligonucleotides:

A person skilled in the art defines labeled oligonucleotides as nucleic acids or analoga to nucleic acids that bear one or several labels suited for the embodiment of this invention. A label is defined as being any molecule or atom capable of generating a measurable, preferably quantifiable signal. According to the definition, labels can be capable of generating measured signals in the form of fluorescence, radioactivity, colorimetry, gravimetry, X-ray deflection or absorption, magnetism, enzymatic activity or similar reactions. Labels, including the methods required for labeling reactions, comprise, but are not limited to, enzyme substrates, radioactive atoms, fluorescence dyes, chromophores, chemiluminescent compounds, electrochemiluminescent compounds, ligands with specific bonding partners or any type of label that can interact with other label ligands, in order to bring about an amplification of, reduction of or change in a measured signal. Preferably, the labels to be used are labels that maintain their stability even at high temperatures, as they are used for example in PCR.

Target Nucleic Acids (Target)

The term “target nucleic acid”, “target” or “target sequence” relates to a sequence section of a heteropolymer nucleic acid or an analogon of a nucleic acid, which is to be amplified, detected, or amplified and detected.

Upstream Probe:

The upstream probe of the probe pair in this context is a heteropolymer nucleic acid or an analogon to a nucleic acid having a partial complementarity to a sub-sequence of the target, which is closer to the 3′ end of the target.

Downstream Probe:

The downstream probe of the probe pair in this context is a heteropolymer nucleic acid or an analogon to a nucleic acid having a partial complementarity to a sub-sequence of the target, which is closer to the 5′ end of the target.

Triplex Structure:

A triplex structure in the sense of this invention is a complex consisting of 2 heteropolymer, single-strand oligonucleotide probes or probes analogous to oligonucleotides as well as single-strand target, wherein hybridisations occur, i.e. the formation of hydrogen bridge bonds between complementary bases both between the probes and the target and between the probes amongst themselves.

Stem Structure:

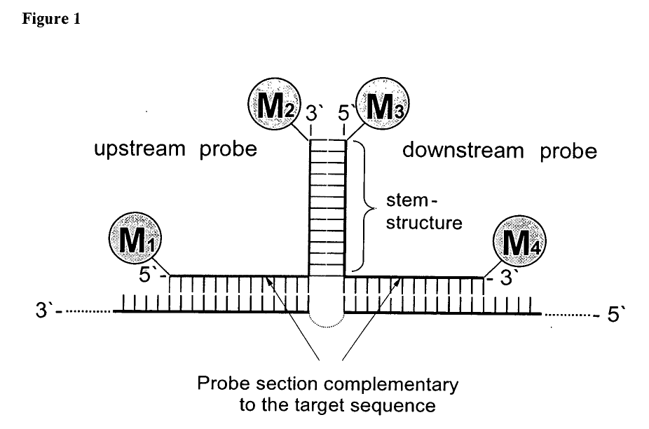

A stem structure is the specific designation for a hybridisation structure within the triplex structure, which exclusively develops between the probes of the probe pair (FIG. 1).

Optimum Hybridisation of the Probes:

An optimum hybridisation of the probes to the target means the formation of a complex triplex structure between probe pairs and target in such a manner that the complex features such a stability as to allow carrying out the method according to the invention as described.

Sufficiently Complementary:

A sufficient complementarity between the probes of the probe pair as well as between the probe pair and the target is given if stable complexes between the probe pair and the target develop to allow carrying out the method according to the invention.

3′-OH blockage:

An oligonucleotide incorporated into a homogeneous detection method acting as a probe has preferably no primer properties according to the state of the art, i.e. the 3′ end of the probe is “blocked” in order to avoid incorporation of the probe into a primer extension product (amplicon). A “blockage” is obtained by either appending non-complementary bases to the 3′ end of the probe or by coupling a chemical group such as biotine or a phosphate group to the 3′ hydroxyle of the last nucleotide. A blockage can moreover be obtained by removing the 3′ OH or by using a nucleotide, such as didesoxynucleotide that generally lacks in 3′ OH groups.

Tₘ Value:

The stability of hybridising nucleic acid duplicates is measured by the “melting temperature” (Tₘ). The Tₘ of a specific nucleic acid duplex structure is the temperature at which 50% of the base pairs within a duplex have dissociated.

Heteropolymer Target Sequence:

A heteropolymer target sequence means any possible target with any possible sequence of bases or analoga to bases, which either follows a natural sequence, such as genomic DNA or RNA, cDNA, or which is of semi-synthetic or synthetic origin.

Heteropolymer Primer Pair:

A heteropolymer primer pair is defined as being a pair of any oligonucleotides with any sequence of bases or analoga to bases which are either of natural origin or which feature a semi-synthetic or synthetic target sequence or which are manufactured synthetically and show a specificity to a natural sequence, such as genomic DNA or RNA, cDNA. A primer serves as initiation point for the synthesis of long, complementary nucleic acid strands, if such primer is used under such catalysing conditions that allow a synthesis of the primer extension products complementary to the target nucleic acid. These conditions are given if there are, at the same time, different desoxyribonucleoside triphosphates and an agent inducing polymerisation, such as a DNA polymerase or reverse transcriptase, a buffer with a suited pH value, cofactors and ion strength, as well as a suited room temperature.

1. Method for the detection of target nucleic acids or analoga to nucleic acids (targets) or for the detection of genetic polymorphisms, wherein at least one pair of labeled oligonucleotides or analoga to oligonucleotides (probes) or at least on labeled probe triplet are used, wherein the upstream probes proportionally hybridise to the target with its 5′ end and proportionally hybridises to the 5′ end of the downstream probe with its 3′ end, as well as the 3′ end of the downstream probe proportionally hybridises to the target again, this resulting in a triplex structure formed between the target nucleic acid and a probe pair.

2. Method according to claim 1, wherein a sufficient hybridisation of the probes to the target occurs only with the probe pair or probe triplet, however not with the individual probes.

3. Method according to claim 1, wherein the sequence sections of the upstream and downstream probes proportionally capable of target hybridisation hybridise to adjacent sequences of the target, and in that simultaneously a stabilising “stem structure” is formed between the 3′ end of the upstream probe and the 5′ end of the downstream probe.

4. Method according to claim 1, wherein ligands can be present both at the 5′ end and at the 3′ end of the probes, and in that there is a total of 4 different loci available for different combinations of labels.

5. Method according to claim 1, wherein the upstream probe is labeled with one ligand acting as a reporter and one ligand acting as a quencher, respectively and features no 3′-OH blockage, whilst the downstream probe is unlabeled and blocked at the 3′ OH.

6. Method according to claim 1, wherein the probe triplet consists of 2 upstream or downstream probes with different ligands, the sequences of which differ at least 1 base, and a related upstream or downstream probe, which is capable of developing a stems with the upstream or downstream probes complementary in the stem area and pertaining to the probe triplet, wherein a hydrolysable triplex structure between upstream probe, downstream probe and target is developed only if there is sufficient complementarity between the target-hybridising probe section and at least one of the probes and the target.

7. Method according to claim 6, wherein in the probe triplet, the two upstream probes are labeled with two ligands emitting at different wave lengths and acting as a reporter as well as with a ligand acting as a quencher, and feature no 3′-OH blockage, whilst the downstream probe is unlabeled and blocked at the 3′-OH.

8. Method according to claim 1, wherein the triplex structure formed between the probe pair and the target is either capable of creating a measured signal directly through the hybridisation event, or in that such measured signal is generated only after partial hydrolysis of the probe pair through an enzyme, preferably a polymerase, which is also contained in the homogeneous assay.

9. Probe pair for the detection of target nucleic acids (targets), wherein the 5′ end of the upstream probe is sufficiently complementary to a sub-sequence of the targets and wherein the 3′ end of this probe is sufficiently complementary to the 5′ end of the downstream probe, and wherein the 3′ of the downstream probe is again sufficiently complimentary to the 5′-end of the upstream probe and the 3′ end of this probe is again sufficiently complementary to another sub-sequence of the targets.

10. Probe pair according to claim 9, wherein either one or both probes have no 3′-OH-blockage.

11. Probe pair according to claims 9, wherein it comprises either the sub-sequences 5′-AGTCGGAACCTT-3′ (SEQ ID NO: 13) and 5′-AAGGTTCCGACT-3′ (SEQ ID NO: 14), or 5′-AGTCGGAAC-3′ and 5′-GTTCCGACT-3′, or other suited substances developing a stem structure.

12. Probe pair according to claim 9, wherein one or both probes bear a label at the 5′ end and/or at the 3′ end.

13. Probe pair according to claim 12, wherein fluorescence dyes and/or quencher dyes are used for labeling.

14. Probe pair according to claim 12, wherein the upstream probe is labeled at the 3′ end and the downstream probe at the 5′ end with a donor dye and an acceptor dye, respectively.

15. Method for the manufacture of a probe pair according to claim 9, comprising the following steps:
Breakdown of a know or unknown oligonucleotide probe sequence compatible with the target and having a length of preferably 1 5-40 bases into two partial probe sequences.
Supplementing of the partial probe sequences each by a sub-sequence according to claim 8 developing a stem structure between the probes of the probe pair.
Coupling of a ligand to the individual probes, which is capable of generating a measurable, preferably quantifiable measured signal in the form of fluorescence, radioactivity, colorimetry, gravimetry, X-ray deflection or X-ray absorption, magnetism, enzymatic activity or similar process (label).

16. Method according to claim 15, wherein both the partial sequence probes hybridised to the target amongst themselves and the complete probes of the probe pair each show similar Tm values.

17. Probe triplet for the detection of genetic polymorphisms, comprising 2 upstream or downstream probes, the sequences of which differ in at least 1 base, and a related upstream or downstream probe.

18. Probe triplet according to claim 17, wherein the two upstream probes are labeled with two ligands emitting at different wave lengths and acting as a reporter, as well as a ligand acting as a quencher, and feature no 3′-OH blockage, whilst the downstream probe is unlabeled and blocked at the 3′-OH.

19. Test kit for carrying out a method according to claim 1, comprising at least one labeled probe pair or probe triplet according to claim 9, at least one primer pair, at least one heteropolymer target sequence, at least one enzyme as well as one related set of reagents.

20. A Method for qualitative and/or quantitative detection of any heteropolymer target nucleic acids or analoga to nucleic acids or for the detection of genetic polymorphisms using the probe pair according to claim 9 or the probe triplet according to the claim 17.

21. A Method for qualitative and/or quantitative detection of any heteropolymer target nucleic acids or analoga to nucleic acids or for the detection of genetic polymorphisms using the test kit according to claim 19.
